(19) United States
(12) Patent Application Publication
(10) Pub. No.: US 2022/0167680 A1
(43) Pub. Date: Jun. 2, 2022

(54) VAPORIZER OVENS, VAPORIZERS, AND RELATED METHODS

(71) Applicant: 1769474 ALBERTA LTD., Edmonton (CA)

(72) Inventors: Tyler Kibler, Edmonton (CA); Steven Fyke, Edmonton (CA); Thai Nguyen, Edmonton (CA); Jason Griffin, Edmonton (CA); Ankit Sharma, Edmonton (CA)

(73) Assignee: 1769474 ALBERTA LTD., Edmonton, AB (CA)

(21) Appl. No.: 17/439,739

(22) PCT Filed: Mar. 13, 2020
(86) PCT No.: PCT/CA2020/050344

§ 371 Date: Sep. 15, 2021

Related U.S. Application Data
(60) Provisional application No. 62/818,850, filed on Mar. 15, 2019.

(51) Int. Cl. A24F 40/49; A24F 40/46; A24F 7/00; A24F 40/48; A24F 40/57; A24F 40/70

(52) U.S. Cl. CPC A24F 40/49; A24F 40/46; A24F 40/70; A24F 40/48; A24F 40/57; A24F 7/00

(57) ABSTRACT

Vaporizer ovens and related vaporizers are provided. The vaporizer oven can comprise a body comprising a vaporizing chamber with an opening and a removable closure member that can engage the body to close the opening. The body can comprise a first locking component and the closure member can comprise a second locking component that interlocks with the first locking component. In some embodiments, a biasing element can be disposed in one of the body or the closure member to exert a biasing force to maintain the closure member in a locked position. A related vaporizer can comprise a housing that receives the vaporizer oven. Related methods for making a vaporizer oven and a vaporizer are also provided.

### RELATED APPLICATION

The present application claims priority to U.S. Provisional Patent Application No. 62/818,850, filed Mar. 15, 2019, the entire contents of which are incorporated by reference herein.

### FIELD OF THE DISCLOSURE

The present disclosure relates to equipment used for vaporizing a material to emit a vapor for inhalation. More particularly, the present disclosure relates to vaporizer ovens and related vaporizers for personal use.

### BACKGROUND

Vaporizers typically heat materials to create a vapor containing one or more desired extracted ingredients. Materials typically vaporized include: dried plant material such as herbs; liquid-based compositions such as oils; and wax-based compositions, etc. Such materials may be referred to as “vaporizing materials” herein. The vapor may then be delivered to a user by inhalation during a “vaping” session.

Portable vaporizers for personal use may comprise an oven, for example a dry herb oven that can vaporize a portion of plant material to emit a vapor. Vaporizer ovens typically comprise an oven body with a vaporizing chamber for receiving the material to be vaporized and a closure member such as a cap for retaining the material in the chamber.

Conventional vaporizer ovens may have closure members that are easily removable to provide access to the vaporizing material therein. However, users must be careful that the vaporizer oven is not opened by a child or another unauthorized user. It is also undesirable for the oven to be inadvertently opened by the user during or between vaping sessions which may cause the vaporizing material to be spilled or the closure means to be misplaced.

### SUMMARY

In one aspect, there is provided a vaporizer oven comprising: a body comprising a vaporizing chamber, an opening to the vaporizing chamber, and a first locking component; and a removable closure member that removably engages the body to close the opening, the closure member comprising a second locking component that interlocks with the first locking component when the closure member engages the body; wherein the closure member is movable when engaged to the body between a locked position in which the first and second locking components are interlocked and the closure member is secured to the body, and an unlocked position in which the first and second locking components are disengaged and the closure member is removable from the body.

In some embodiments, one of the first and second locking components comprises a projection and the other of the first and second locking components comprises a recess, the projection being received into the recess to interlock the first and second locking components when the closure member engages the body in the locked position.

In some embodiments, the closure member rotatably engages the body and wherein the closure member is rotatable between a first rotational position in which the projection is received within the recess and the first and second locking components are interlocked, and a second rotational position in which the projection is displaced from the recess and the first and second locking components are disengaged.

In some embodiments, the recess comprises at least one axial boundary that limits axial movement of the projection when the first and second locking components are interlocked, thereby limiting axial movement of the closure member away from the body when the closure member is in the first rotational position.

In some embodiments, the vaporizer oven further comprises at least one biasing element disposed in one of the body and the closure member, the at least one biasing element exerting a biasing force to maintain the closure member in the first rotational position.

In some embodiments, at least a portion of the recess is disposed between a first rotational stop and a second rotational stop; and wherein the biasing force of the biasing element holds the projection within the recess between the first and second rotational stops to maintain the closure member in the first rotational position.

In some embodiments, the at least one biasing element is compressible, and compression of the biasing element provides the biasing force.

In some embodiments, the closure member is axially movable with respect to the body and wherein axial movement of the closure member with respect to the body compresses the biasing element to overcome the biasing force.

In some embodiments, the first locking component is integral to the body and the second locking component is integral to the closure member.

In some embodiments, the closure member comprises a mouthpiece portion having a suction opening, the suction opening in fluid communication with the vaporizing chamber when the closure member is engaged with the body.

In some embodiments, the closure member further comprises an inner sleeve defining an internal conduit therethrough, the internal conduit in fluid communication with the suction opening and the vaporizing chamber when the closure member is engaged with the body.

In some embodiments, the closure member further comprises a helical insert received within the internal conduit, the helical insert defining approximately spiral airflow path between the vaporizing chamber and the suction opening.

In some embodiments, the inner sleeve is colored to indicate a characteristic of a vaporized material to be vaporized in the vaporizing chamber.

In some embodiments, the vaporizer oven further comprises a heating means operatively connected to the vaporizing chamber, the heating means activatable to heat the vaporizing chamber.

In some embodiments, the vaporizer oven further comprises an activation control mechanism, operatively connected to the heating means, wherein the activation control mechanism disables activation of the heating means when the closure member is disengaged from the body.

In some embodiments, the activation control mechanism comprises a sensor in one of the body and the closure member that senses when the closure member is disengaged from the body.

In some embodiments, the activation control mechanism comprises an electrical switch that is open when the closure member is disengaged from the body and closed when the closure member engages the body.

In another aspect, there is provided a vaporizer comprising: embodiments of the vaporizer oven described herein; and a housing that engages the vaporizer oven, the housing having a cavity that at least partially receives the vaporizer oven.

In some embodiments, the cavity is defined by an inner wall that covers at least a portion of the vaporizer oven when the vaporizer oven is at least partially received into the cavity.

In some embodiments, the inner wall of the cavity covers at least a portion of the closure member of the vaporizer oven to prevent movement of the closure member between the locked and unlocked positions.

In some embodiments, the housing further comprises a slidable sidewall that engages the vaporizer oven to longitudinally slide the vaporizer oven into and out of the cavity.

In some embodiments, the housing is configured to receive the vaporizer oven into the cavity with the vaporizer oven in an operational position in which the closure member is directed outward from the cavity, and receive the vaporizer oven into the cavity with the vaporizer oven in a non-operational position in which the closure member is directed inward into the cavity.

In another aspect, there is provided a method for making a vaporizer oven, comprising: providing a body comprising a vaporizing chamber and a first locking component; and providing a closure member that removably engages the body, the closure member comprising a second locking component that interlocks with the first locking component.

In some embodiments, the method further comprises engaging the closure member with the body in one of a locked position and an unlocked position.

In another aspect, there is provided a method for making a vaporizer, comprising: providing a vaporizer oven comprising a body having a vaporizer chamber and a closure member that engages the body; and providing a housing comprising a cavity that removably receives the vaporizer oven in an operational position and a non-operational position.

In some embodiments, the method further comprises inserting the vaporizer oven into the cavity in one of the operation position and the non-operational position, wherein inserting the vaporizer oven comprises sliding the vaporizer oven longitudinally with respect to the housing.

Other aspects and features of the present disclosure will become apparent, to those ordinarily skilled in the art, upon review of the following description of the specific embodiments of the disclosure.

### BRIEF DESCRIPTION OF THE DRAWINGS

The present disclosure will be better understood having regard to the drawings in which:

FIG. 1A is a perspective view of an example vaporizer oven, according to some embodiments, showing an embodiment of a closure member engaged with the body of the vaporizer oven;

FIG. 1B is a perspective view of the vaporizer oven of FIG. 1A showing the closure member moved towards the body;

FIG. 1C is a perspective view of the vaporizer oven of FIG. 1A showing the closure member rotated with respect to the body;

FIG. 1D is a perspective view of the vaporizer oven of FIG. 1A showing the closure member disengaged from the body;

FIG. 1E is a perspective view of the body of the vaporizer oven of FIG. 1A with the closure member removed;

FIG. 2 is an enlarged perspective view of the body of the vaporizer oven shown in FIG. 1E;

FIGS. 3A and 3B are enlarged perspective and side views, respectively, of the closure member of the vaporizer oven shown in FIGS. 1A-1D;

FIG. 4A is an exploded, perspective view of the vaporizer oven of FIG. 1A;

FIGS. 4B-4D are perspective views of various subassemblies of the vaporizer oven of FIG. 4A;

FIG. 5 is a cross-sectional view of the vaporizer oven of FIG. 1A;

FIG. 6A is an exploded, perspective view of another example vaporizer oven, according to some embodiments;

FIG. 6B is a perspective view of the vaporizer oven of FIG. 6A, shown partially assembled;

FIG. 6C is a cross-sectional view of the vaporizer oven of FIG. 6A;

FIGS. 7A and 7B are exploded, perspective views of another example vaporizer oven, according to some embodiments;

FIG. 7C is a cross-sectional view of the vaporizer oven of FIGS. 7A and 7B;

FIG. 8 is a perspective view of another example vaporizer oven, according to some embodiments;

FIGS. 9A-9C are perspective views of an example vaporizer, according to some embodiments, including the vaporizer oven of FIG. 1A;

FIGS. 10A-10C are perspective views of the vaporizer of FIGS. 9A-9C with the vaporizer oven in a non-operational position;

FIG. 11 is perspective view of the vaporizer of FIG. 9C, showing the bottom of the vaporizer;

FIG. 12 is a functional block diagram of an example vaporizer with an activation control mechanism, according to some embodiments;

FIG. 13 is a functional block diagram of another example vaporizer, according to some embodiments;

FIG. 14 is a flowchart of a method for making a vaporizer oven, according to some embodiments; and

FIG. 15 is a flowchart of a method for making a vaporizer, according to some embodiments.

### DETAILED DESCRIPTION

Generally, the present disclosure provides a vaporizer oven and related vaporizer. The vaporizer oven may comprise: a body comprising a vaporizing chamber, an opening to the vaporizing chamber, and a first locking component; and a removable closure member that engages the body to close the opening, the closure member comprising a second locking component that interlocks with the first locking component when the closure member engages the body. In some embodiments, the closure member is movable when engaged to the body between: a locked position in which the first and second locking components are interlocked and the closure member is secured to the body; and an unlocked position in which the first and second locking components are disengaged and the closure member is removable from the body. Also provided is a related vaporizer comprising a housing that can receive the vaporizer oven. Related methods for making a vaporizer oven and a vaporizer are also provided.

It is to be understood that directional or relative terms such as “up”, “down”, “upward”, “downward”, “forward”, “front”, “rearward”, “back”, “vertical”, “horizontal”, “side”, “top”, “bottom” and the like are used for ease of description and illustrative purposes, and embodiments are not limited to a particular orientation of the vaporizer ovens or vaporizers described herein during use or normal operation.

As used herein and in the appended claims, the singular forms “a”, “an”, and “the” include plural referents unless the context clearly dictates otherwise.

The present disclosure relates to vaporizer ovens and related vaporizers and methods. The term “vaporizer” may refer to any device configured to vaporize a material to produce a vapor to be inhaled by a user. The term “portable vaporizer apparatus” may refer to any vaporizer apparatus that is able to be carried by a user for portable use, such as vaporizer pens, portable dry herb vaporizers, electronic cigarettes, etc. Portable vaporizer apparatuses may be configured for use with one or more vaporizing materials including dried plant material such as herbs, liquid-based compositions such as oils, wax-based compositions, etc. The term “vaporizer oven” may refer to an apparatus used with a vaporizer, or forms part of a vaporizer, that comprises a chamber therein in which the material is vaporized.

In some embodiments, the vaporizer oven and/or related vaporizer can comprise one or more child-lock safety features. As used herein, the term “child-lock safety feature” may refer to any feature that is designed to help prevent a child or an unauthorized user from performing any action that poses a danger to the child or the unauthorized user. As used herein, the term “unauthorized user” may refer to any person that does not have authorization to use a vaporizer oven or vaporizer including, for example, persons having diminished mental capacity because of illness or disability such as a mental health problem, dementia, or learning disability. One or more safety features may also function to prevent inadvertent or accidental opening of the vaporizer oven by the user during or between “vape” sessions.

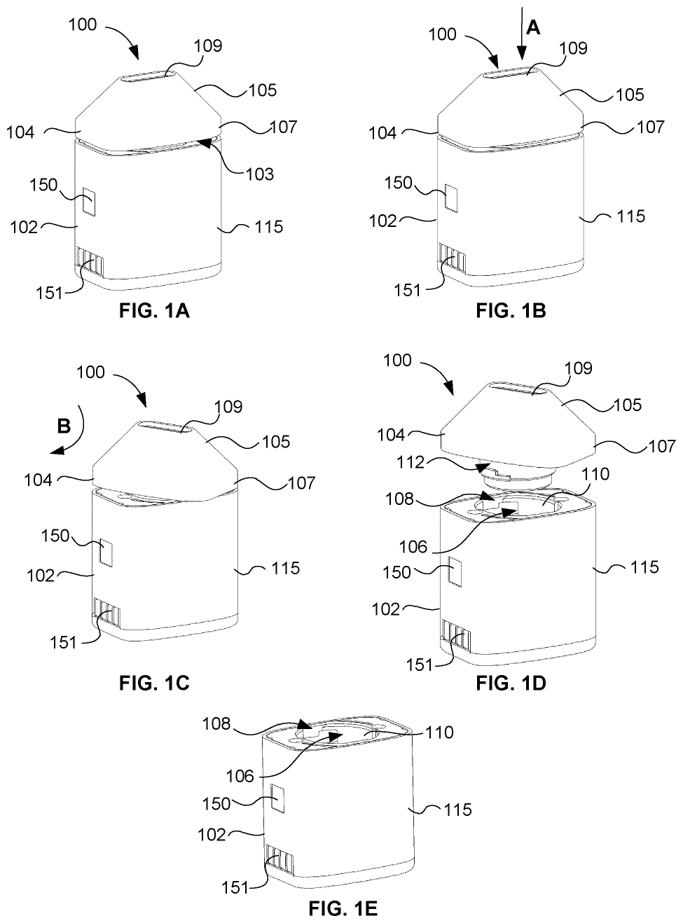

An example vaporizer oven 100 will be described with reference to FIGS. 1A to 1E. The vaporizer oven 100 may comprise a body 102 and a removable closure member 104 (removed in FIG. 1E) configured to engage the body 102.

The body 102 may be any suitable size and shape known in the art. In some embodiments, the body 102 may have a non-circular cross-section across its width. For example, the body 102 may have an approximately rectangular or ovoid width-wise cross-section. In other embodiments, the body 102 may have an approximately circular width-wise cross-section or a width-wise cross-section of any other suitable shape.

The body 102 may comprise a vaporizing chamber 106. The vaporizing chamber 106 may be configured to hold a material to be vaporized. In some embodiments, the material to be vaporized may be a plant material. In some embodiments, the plant material may be a dried herb. Embodiments are not limited to the specific types of vaporizing materials described herein.

The body 102 may also comprise a heating means (not shown) operatively connected to the vaporizing chamber 106 and activatable to vaporize at least a portion of the material therein. In some embodiments, the heating means may comprise a heated element such as a coiled heating element. The coiled heated element may comprise, for example, one or more metal, ceramic, or glass heating coils. In other embodiments, the heating element may comprise any other element configured to generate sufficient heat to vaporize the vaporizing material.

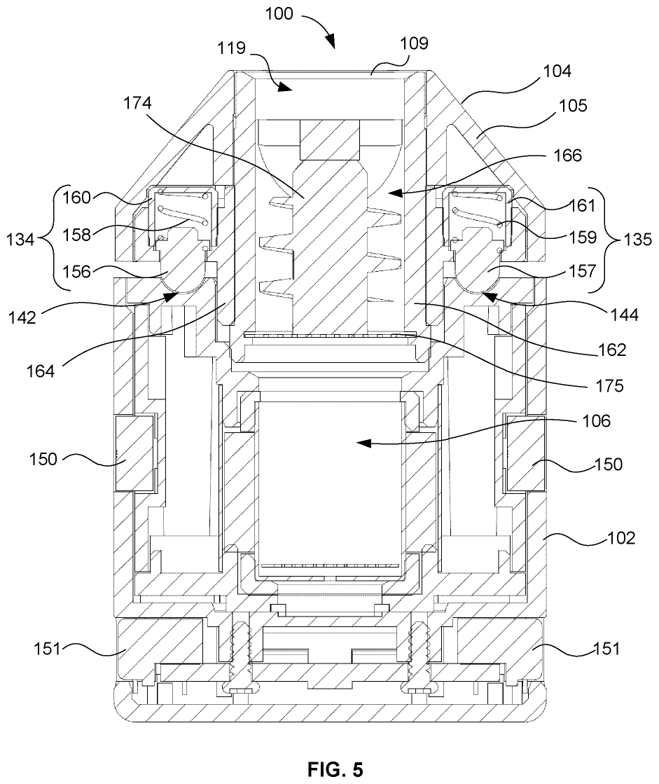

The heating element may be operatively connectable to a power source, such as a battery (not shown), in a vaporizer housing as described in more detail below. In some embodiments, the vaporizer oven 100 may comprise one or more electrical connectors 151 configured to engage a cooperating electrical connector(s) in the vaporizer housing to operably connect the power source to the heating element. In some embodiments, a respective electrical connector 151 may be provided on each side of the vaporizer oven 100 (as shown in FIG. 5). In some embodiments, the heating element, when provided with power from the power source, may provide heat to the vaporizing materials contained within the vaporizing chamber 106 to emit a vapor. In some embodiments, the body 102 may further comprise at least one magnet 150 to help maintain the vaporizer oven 100 in the vaporizer housing, as described in more detail below.

The body 102 may further comprise an opening 108 to the vaporizing chamber 106. The body 102 may have an inner wall 110 and an outer wall 115, the inner wall 110 defining the opening 108. In this embodiment, the opening 108 may be a circular opening. In other embodiments, the opening 108 may be any other suitable shape. The opening 108 may provide access to the vaporizing chamber 106 to allow the user to add a material to be vaporized and/or to remove residual material following a “vape” session.

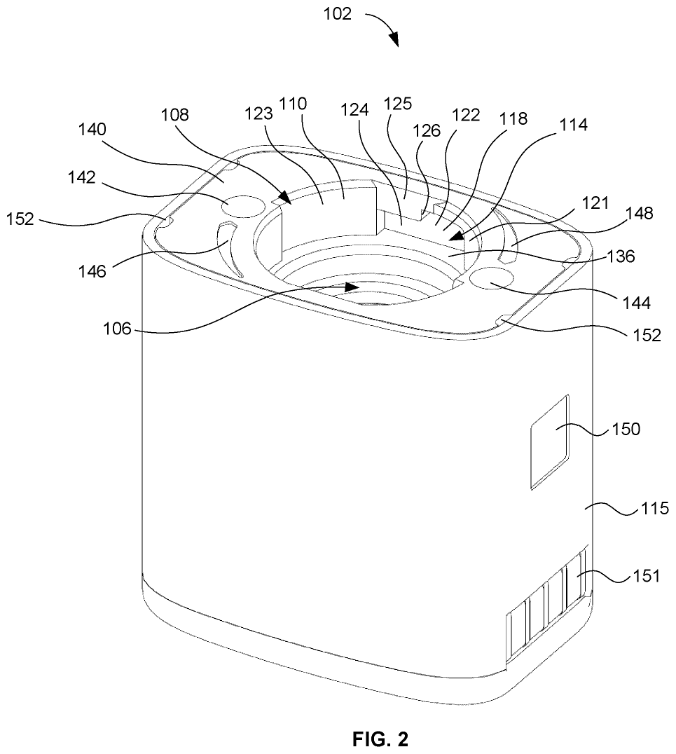

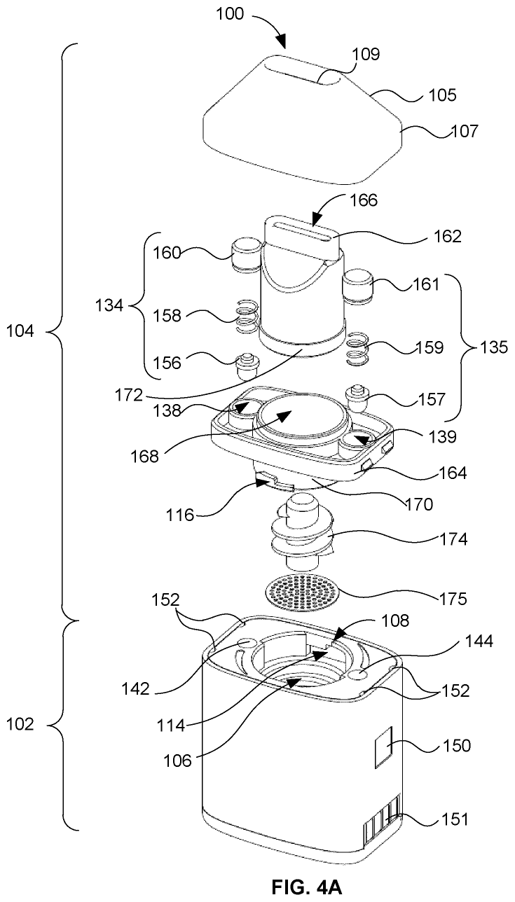

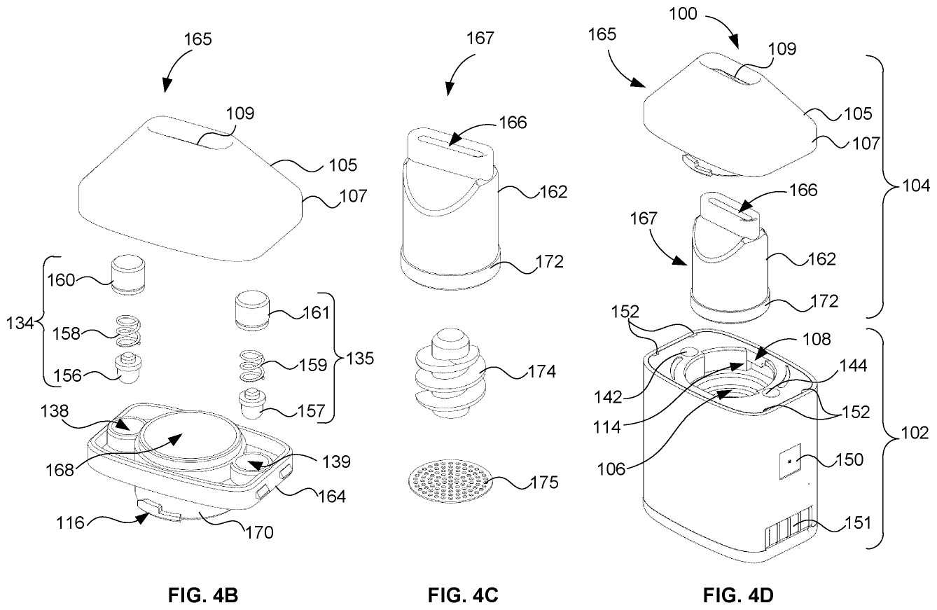

In some embodiments, the body 102 may further comprise at least one airflow opening 152 (shown in FIGS. 2 and 4A) in fluid communication with the vaporizing chamber 106. In some embodiments, the body 102 may comprise a plurality of airflow openings 152 such as, for example, the airflow openings 152 shown in FIGS. 2 and 4A.

The closure member 104 may engage the body 102 to close the opening 108, for example, when the vaporizer oven 100 is being used by a user for a “vape” session. The closure member 104 may be disengaged from the body 102 as needed for the user to access the opening 108 to the vaporizing chamber 106. The term “disengaged” in this context may refer to any position of the closure member 104 in which the closure member 104 does not close the opening 108 to vaporizing chamber 106, although the closure member 104 may still be in contact with the body 102.

In some embodiments, the closure member may comprise a mouthpiece portion 105 having a suction opening 109. The suction opening 109 of the mouthpiece portion 105 may be in fluid communication with the vaporizing chamber 106 when the closure member 104 is engaged with the body 102. In some embodiments, the closure member 104 may comprise a longitudinal conduit 119 (shown in FIG. 5) therethrough to fluidly connect the suction opening 109 to the vaporizing chamber 106 when the closure member 104 is engaged with the body 102. During a “vape” session, a user may position his or her mouth on mouthpiece portion 105 and inhale, which may cause vapor contained within the vaporizing chamber 106 to be drawn through the suction opening 109 and into the user's mouth and lungs for consumption.

The mouthpiece portion 105 may further comprise a base section 107 configured to engage the body 102. In some embodiments, the base section 107 may have a width-wise cross-section approximately the same size and shape as that of the body 102 such that the base section 107 and the body 102 are substantially flush when the closure member 104 is engaged with the body 102. In some embodiments, the mouthpiece portion 105 may taper from the base section 107 towards the suction opening 109. In other embodiments, the mouthpiece portion 105 may be any other suitable shape.

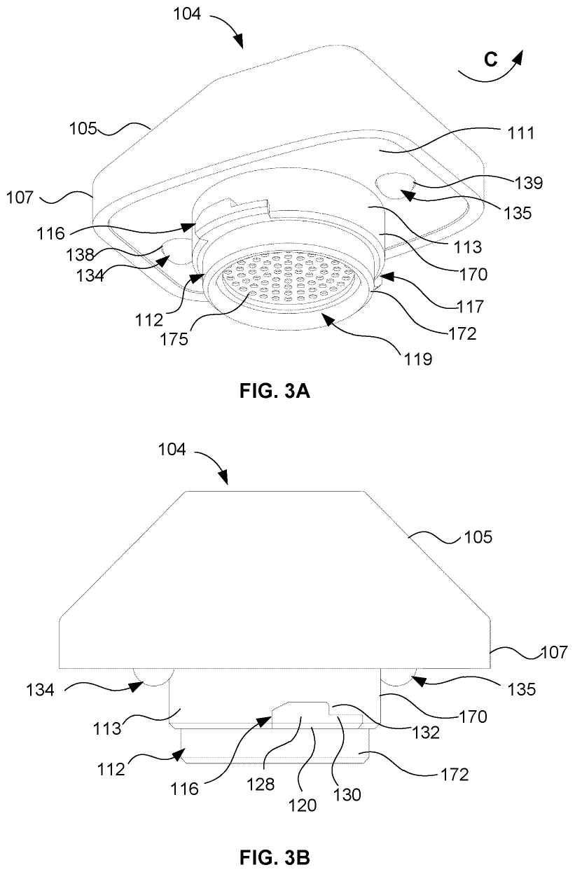

The closure member 104 may further comprise a plug portion 112 extending from the mouthpiece portion 105. The plug portion 112 may have an outer wall 113 (shown in FIGS. 3A and 3B). As shown in FIGS. 3A and 3B, in some embodiments, the plug portion 112 may comprise an upper plug portion 170 and a lower plug portion 172. The plug portion 112 (more particularly, the upper plug portion 170) may be received into the opening 108 of the vaporizing chamber 106 when the closure member 104 is engaged with the body 102, thereby closing the opening 108. The longitudinal conduit 119 may extend through the plug portion 112 such that the fluid connection between the suction opening 109 and the vaporizing chamber 106 may be maintained when the plug portion 112 is received into the opening 108.

Assembly of the closure member 104 will be discussed in more detail with reference to FIGS. 4A-4D and 5. In some embodiments, the closure member 104 may further comprise an inner sleeve 162 (also referred to as a “mouthpiece nozzle”) and an inner support structure 164. The inner sleeve 162 and inner support structure 164 may be partially received into the mouthpiece portion 105. The inner sleeve 162 may have a first internal conduit 166 extending therethrough. The first internal conduit 166 may be in fluid communication with the suction opening 109 and the vaporizing chamber 106 when the closure member 104 is engaged with the body 102. The inner support structure 164 may have a second internal conduit 168 extending therethrough. When the closure member 104 is assembled, the inner sleeve 162 may be received into the second internal conduit 168 of the inner support structure 164 and extend past the inner support structure 164, such that the lower end of the inner sleeve 162 forms the lower plug portion 172 and the lower end of the inner support structure 164 forms the upper plug portion 170.

In some embodiments, the inner sleeve 162 may be comprised of a flexible material, such as rubber. In other embodiments, the inner sleeve 162 may be comprised of any other suitable material. In some embodiments, the inner sleeve 162 may be colored (for example by using a colored rubber material) to indicate to the user a characteristic of the vaporizing material to be vaporized in the vaporizing chamber 106. For example, where the vaporizing material is a plant material, the color of the inner sleeve 162 may indicate the plant species or strain, the chemical composition of the plant material, the form of the plant material (e.g. dried, liquid, or wax-based, etc.), or any other relevant characteristic of the plant material.

In some embodiments, a helical insert 174 may be received into the first internal conduit 166 of the inner sleeve 162. The helical insert 174 may define an approximately spiral airflow path from the vaporizing chamber 106 to the suction opening 109 (when the closure member 104 is engaged with the body 102), thereby increasing the overall length of the airflow path of the vapor generated from the vaporizing material. Lengthening the airflow path may reduce the temperature of the vapor reaching the user's mouth such that the vapor is a comfortable temperature for the user. In other embodiments, inserts of alternative shapes and/or configurations may be provided to lengthen the airflow path of the vapor and embodiments are not limited to helical inserts. For example, the insert may be an approximately cylindrical shaped conduit and/or the insert may be perforated, etc.

In some embodiments, the closure member 104 may further comprise a screen 175 extending across the first internal conduit 166 to prevent vaporizing material or residual material following vaporization from being drawn from the vaporizing chamber 106 into the suction opening 109. In some embodiments, the screen 175 comprises a mesh filter having a suitable mesh size to prevent the vaporizing material and residual material from being drawn therethrough.

In some embodiments, the closure member 104 may be assembled in two or more subassemblies as shown in FIGS. 4B-4D. As shown in FIG. 4B, a first subassembly 165 may comprise the mouthpiece portion 105, the first and second biasing elements 134 and 135, and the inner support structure 164. As shown in FIG. 4C, a second subassembly 167 may comprise the inner sleeve 162, the helical insert 174, and the screen 175. The first and second subassemblies 165 and 167 may then be assembled to form the final closure member 104 to engage the body 102, as shown in FIG. 4D.

In some embodiments, the second subassembly 167 may be removable and replaceable such that the user may separate the second subassembly 167 from the first subassembly 165 and disassemble the second assembly 167 into its component parts. The inner sleeve 162 of the second assembly 167 may thereby be replaced with another inner sleeve (e.g. of a different color) as desired and reassembled with the helical insert 174 and screen 175. Alternatively, the entire second assembly 167 may be replaced with a new second assembly comprising a new inner sleeve, helical insert, and screen.

In alternative embodiments, the closure member 104 may engage a first end of the body 102 and a separate mouthpiece member with a suction opening may engage an opposed end of the body 102 such that the suction opening of the separate mouthpiece member is in fluid communication with the vaporizing chamber 106, while the closure member 104 closes an opposed end of the vaporizing chamber 106.

Referring to FIGS. 2, 3A and 3B, the body 102 may comprise a first locking component 114 and the closure member 104 may comprise a second locking component 116 configured to interlock with the first locking component 114. The closure member 104 may thereby have a locked position in which the first and second locking components 114 and 116 are interlocked and the closure member 104 is secured to the body 102 to close the opening 108. The closure member 104 may also have an unlocked position in which the first and second locking components 114 and 116 are disengaged and the closure member 104 is removable from the body 102.

In some embodiments, the closure member 104 may be movable between the locked position and the unlocked position when engaged with the body 102. In some embodiments, movement of the closure member 104 between the locked and unlocked positions may comprise rotation of the closure member 104. The closure member 104 may rotatably engage the body 102 and be rotatable between a first rotational position (as shown in FIGS. 1A and 1B) in which axial movement of the closure member 104 away from the body is limited and a second rotational position (as shown in FIG. 1C) in which axial movement of the closure member 104 away from the body is permitted and the closure member 104 can be removed.

In some embodiments, the first locking component 114 may be integral to the body 102. In some embodiments, the second locking component 116 may be integral to the closure member 104. As shown in FIG. 2, the first locking component 114 in this embodiment may be integral to the inner wall 110 of the body 102 defining the opening 108. As shown FIGS. 3A and 3B, the second locking component 116 in this embodiment may be integral to the outer wall 113 of the plug portion 112 of the closure member 104 (more particularly, the upper plug portion 170). In other embodiments, the first and/or second locking components 114 and 116 may be coupled to the body 102 and closure member 104, respectively, by any suitable coupling means.

As shown in FIG. 2, the first locking component 114 may comprise a recess 118 in the inner wall 110. In some embodiments, the inner wall 110 of the body 102 may comprise an annular shelf portion 136, extending radially inwards, between the opening 108 and the vaporizing chamber 106. The annular shelf portion 136 may form a lower axial boundary of the recess 118. The inner wall 110 may further comprise an upper portion 125 forming the upper axial boundary of the recess 118.

The recess 118 may comprise a first recess section 122 axially offset from second recess section 124, thereby forming a rotational stop 126. The recess 118 may be disposed between a first inner wall section 121 and a second inner wall section 123, with the first inner wall section 121 projecting further radially inwards than the second inner wall section 123. The first inner wall section 121 may therefore function as a second rotational stop.

As shown in FIGS. 3A and 3B, the second locking component 116 may comprise a projection 120 that may be received into the recess 118 to interlock the first and second locking components 114 and 116. When the projection 120 is received into the recess 118, the first and second locking components 114 and 116 may be interlocked and the closure member 104 may be in the first rotational position such that axial movement of the closure member 104 away from the body 102 is limited by the upper axial boundary of the recess 118 (i.e. by the upper portion 125 of the inner wall 110). As described in more detail below, when the closure member 104 is rotated to the second rotational position, the projection 120 may be displaced from the recess 118 such that the first and second locking components are disengaged and axial movement of the closure member 104 away from the body 102 is permitted and the closure member 104 can be removed from the body 102.

The projection 120 may comprise a first projection section 128 axially offset from a second projection section 130, thereby forming a stop abutment section 132. The first projection section 128 may be received in the first recess section 122 and second projection portion 130 may be received in the second recess portion 124 to interlock the projection 120 with the recess 118. When the first and second locking components 114, 116 are interlocked, the stop abutment section 132 may abut the stop 126 to prevent rotation of the closure member 104 with respect to the body 102 (as indicated by arrow C in FIG. 3) when the closure member 104 is engaged with the body 102. Also, when the first and second locking components are interlocked, the first inner wall section 121 may act as a second rotational stop to prevent rotation of the closure member 104 in the opposite direction than that indicated by arrow C, when the closure member 104 is engaged with the body 102 in the locked position.

In other embodiments, the first locking component 114 may comprise a projection and the second locking component 116 may comprise a complementary recess. In other embodiments, the first and second locking components 114, 116 may comprise any other suitable types of complementary locking components.

In some embodiments, the body 102 may further comprise a third locking component (not shown) and the closure member 104 may further comprise a fourth locking component 117 (shown in FIG. 3A) that can interlock with the third locking component. The third locking component and the fourth locking component 117 may be rotationally offset from the first and second locking components 114 and 116, respectively. In other embodiments, additional pairs of locking components may also be provided.

In some embodiments, the vaporizer oven 100 may further comprise at least one biasing element that exerts a biasing force to hold the closure member 104 in the locked position. As shown in FIGS. 3A and 3B, in this example, a first biasing element 134 and a second biasing element 135 may be provided. In this embodiment, the first and second biasing elements 134 and 135 may be disposed in the closure member 104. In other embodiments, the first and second biasing elements 134 and 135 may be disposed in the body 102.

In some embodiments, the first and second biasing elements 134 and 135 may be compressible and compression of the first and second biasing elements 134 and 135 may provide the biasing force. As shown in FIGS. 4A, 4C, and 5, the first biasing element 134 may comprise a first protruding element 156, a first spring 158, and a first spring housing 160. The second biasing element 135 may comprise a second protruding element 157, a second spring 159, and a second spring housing 161. In some embodiments, the first and second springs 158 and 159 may comprise coil springs. The coil springs may be made of spring steel, copper, bronze, or another suitable material. Compression of the first and second springs 158 and 159 may thereby provide the biasing force of the first and second biasing elements 134 and 135. However, the first and second biasing elements 134 and 135 are not limited to springs and other mechanisms may be used to exert a biasing force in other embodiments.

The first and second protruding elements 156 and 157 may extend through respective apertures 138 and 139 in the support structure 164 and protrude outward from a bottom surface 111 of the closure member 104, on either side of the plug portion 112, as shown in FIG. 3A. In some embodiments, the first and second protruding elements 156 and 157 may be approximately hemi-spherical in shape. In other embodiments, the first and second protruding elements 156 and 157 may be any suitable shape.

As shown in FIG. 2, the body 102 may comprise a top surface 140 having a first and second recess 142 and 144 configured to receive the first and second protruding elements 156 and 157. In some embodiments, the top surface 140 of the body 102 may further comprise first and second arrow grooves 146 and 148 to indicate to the user which direction to rotate the closure member 104 to disengage or re-engage the closure member 104 with the body 102 as described below. The first and second recesses 142 and 144 may be relatively shallow such that the first and second protruding elements 156 and 157 are only partially received into the first and second recesses 142 and 144 when the closure member 104 is engaged with the body 102 in the locked position. The first and second springs 158 and 159 may thus be partially compressed, thereby providing a biasing force. The biasing force may bias the closure member 104 away from the body 102 thereby maintaining the first projection section 128 of the second locking component 116 in the first recess section 122 of the first locking component 114, between the rotational stop 126 and the inner wall section 121, which acts as a second rotational stop. Thus, the first and second locking components 114, 116 may be maintained in interlocked engagement. The fourth locking component 117 may similarly be maintained in locking engagement with the third locking component. The closure member 104 may thereby be prevented from rotating from the first rotational position to the second rotational position and thus the closure member 104 may be maintained in the locked position.

An example of how the closure member 104 may be disengaged from the body 102 will now be discussed with reference to FIGS. 1A to 1E. As shown in FIG. 1A, when the closure member 104 in this embodiment is engaged with the body 102 in the locked position, a small gap 103 may be present between the closure member 104 and the body 102 to allow a limited range of axial movement of the closure member 104 with respect to the body 102. As shown in FIG. 1B, the closure member 104 may be moved axially towards the body 102 as indicated by arrow A. Axial movement of the closure member 104 towards the body 102 may compress the first and second biasing elements 134 and 135 to overcome the biasing force. The gap 103 between the closure member 104 and the body 102 may be reduced or eliminated. Movement of the closure member 104 towards the body 102 may displace the first projection section 128 from the first recess section 122 such that the stop abutment section 132 is displaced from the stop 126. With the first projection section 128 displaced from the first recess section 122, the closure member 104 may be rotated with respect to the body 102 from the first rotational position to the second rotational position as indicated by arrow B sin FIG. 1C. Rotation of the closure member 104, while the first and second biasing elements 134 and 135 are compressed, may allow the projection 120 to be slid through the second recess portion 124, and out of the recess 118, clearing the stop 126. The first and second locking components 114, 116 may thereby be disengaged (and the third and fourth locking components may be similarly disengaged) and the closure member may now be in the unlocked position. The closure member 104 may then be disengaged from the body 102 as shown in FIG. 1D. As shown in FIG. 1E, the closure member 104 may be removed from the body 102.

A person skilled in the art will recognize that while FIGS. 1A-1E show the closure member 104 being rotated in the direction indicated by arrow B while the body 102 is held stationary, the same effect may be achieved by rotating the body 102 in the reverse direction to arrow B while the closure member 104 is held stationary, or by rotating the closure member 104 and the body 102 in opposite directions at once.

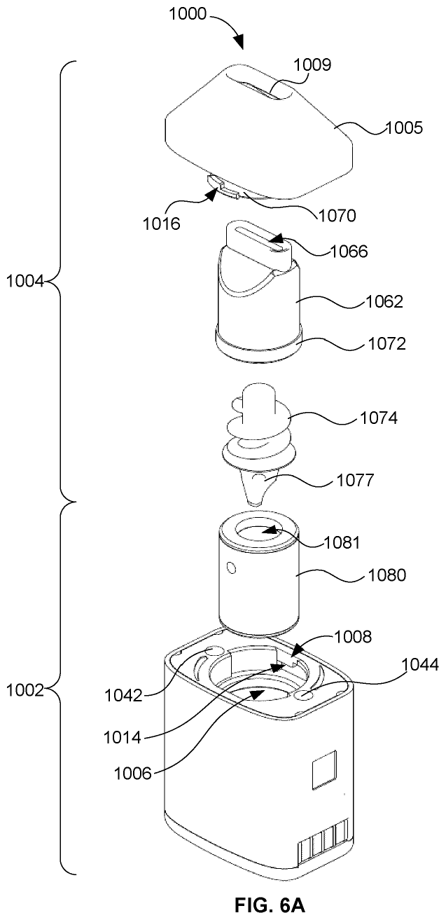

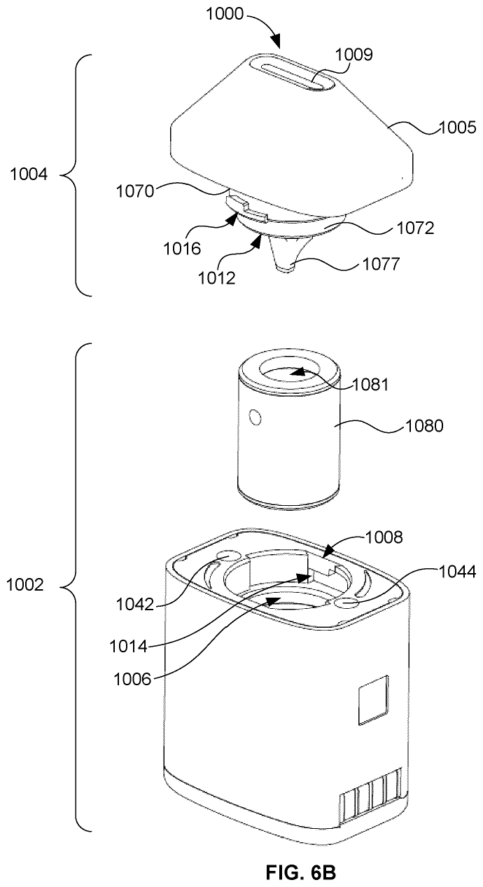

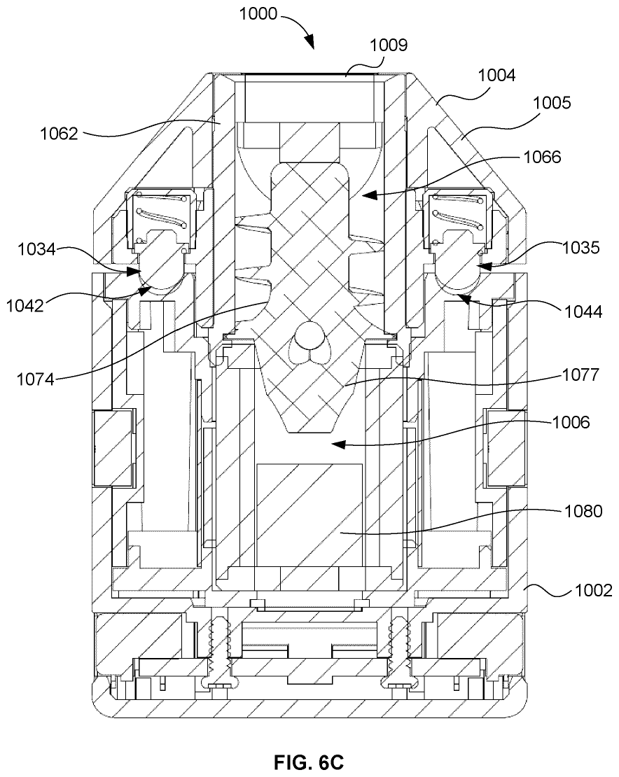

Another example vaporizer oven 1000 will be discussed with reference to FIGS. 6A to 6C. In this embodiment, the vaporizer oven 1000 may comprise a body 1002 and a closure member 1004.

The body 1002 may comprise a vaporizing chamber 1006 and an opening 1008 to the vaporizing chamber 1006. The body 1002 may further comprise a heating means as described above for the vaporizer oven 100.

In some embodiments, the body 1002 may further comprise a removable receptacle 1080 configured to be received into the vaporizing chamber 1006. In some embodiments, the vaporizing chamber 1006 and the receptacle 1080 may each be approximately cylindrical in shape. In other embodiments, the vaporizing chamber 1006 and the receptacle 1080 may each be any other suitable shape such that the receptacle 1080 may be received into the vaporizing chamber 1006. In some embodiments, a magnetic connection may be provided between the receptacle 1080 and an inner wall (not shown) of the vaporizing chamber 1006 to help retain the receptacle 1080 in the vaporizing chamber 1006. In other embodiments, the receptacle 1080 may be held in the vaporizing chamber 1006 by any suitable means.

The receptacle 1080 may define an internal chamber 1081 therein. The internal chamber 1081 may be configured to receive a vaporizing material to be vaporized. In some embodiments, the vaporizing material may be a liquid-based composition, such as an oil, or a wax-based composition, etc. In some embodiments, the receptacle 1080 may be made of a porous material (e.g. ceramic) such that at least a portion of the liquid or wax may be absorbed into the porous material. The heating means in the body 1002 may thereby heat the liquid/wax to generate a vapor therefrom.

Thus, in some embodiments, when a user is desirous of vaporizing a liquid or a wax-based vaporizing material, the receptacle 1080 may be inserted into the vaporizing chamber 1006 and when the user is desirous of vaporizing a dried herb or other solid vaporizing material, the receptacle 1080 may be removed and the vaporizing material may be received directly into the vaporizing chamber 1006.

The closure member 1004 may removably engage the body 1002 to close the opening 1008. The closure member 1004 may comprise a mouthpiece portion 1005 and a suction opening 1009. As shown in FIG. 6B, the closure member 1004 may comprise a plug portion 1012 that is received into the opening 1008 of the body 1002.

The body 1002 may comprise a first locking component 1014. The closure member 1004 may comprise a second locking component 1016 configured to interlock with the first locking component 1014 when the closure member 1004 is engaged with the body 1002. The closure member 1004 may therefore have a locked position and an unlocked position.

In some embodiments, the closure member 1004 may comprise a first biasing element 1034 and a second biasing element 1035 (shown in FIG. 6C). The body 1002 may comprise a respective first recess 1042 and second recess 1044 to at least partially receive the first and second biasing elements 1034 and 1035. The first and second biasing elements 1034 and 1035 may function in a similar manner to the first and second biasing elements 134 and 135 of the vaporizer oven 100.

The plug portion 1012 of the closure member 1004 may comprise an upper plug portion 1070 and a lower plug portion 1072. In some embodiments, the upper plug portion 1070 may extend from the mouthpiece portion 1005. The upper plug portion 1070 may be integral to the mouthpiece portion 1005 or may be coupled to the mouthpiece portion 1005 by any suitable coupling means. In this example, the second locking component may be disposed on the upper plug portion 1070. The mouthpiece portion 1005 may define a first internal conduit 1071 therein (visible in FIG. 6C).

In some embodiments, the closure member 1004 may further comprise a removable inner sleeve 1062, similar to the inner sleeve 162 of the vaporizer oven 100. The inner sleeve 1062 may be partially received into the first internal conduit 1071 of the mouthpiece portion 1005. The inner sleeve 1062 may define a second internal conduit 1066 therethrough in fluid communication with the suction opening 1009 and the vaporizing chamber 1006 when the closure member 1004 is engaged with the body 1002. When the closure member 1004 is assembled, the inner sleeve 1062 may be partially received into the first internal conduit 1071 of the mouthpiece portion 1005 and extend past the mouthpiece portion 1005, such that the lower end of the inner sleeve 1062 forms the lower plug portion 1072, below the upper plug portion 1070.

In some embodiments, a helical insert 1074 may be received into the second internal conduit 1066 of the inner sleeve 1062. The helical insert 1074 may define an approximately spiral airflow path from the vaporizing chamber 1006 to the suction opening 1009. In some embodiments, the helical insert 1074 may further comprise a scooping element 1077 that extends past the inner sleeve 1062 when the helical insert 1074 is received into the second internal conduit 1066.

FIG. 6B shows the assembled closure member 1004. In use, the user may grip the mouthpiece portion 1005 of the closure member 1004 and use the scooping element 1077 to scoop a portion of a liquid or wax-based vaporizing material to be vaporized. As shown in FIG. 6C, when the closure member 1004 is engaged with the body 1002, the scooping element 1077 may extend into the vaporizing chamber 1006, above the receptacle 1080. When the heating means is activated, the liquid/wax on the scooping element 1077 may become less viscous and may drip into the internal chamber 1081 of the receptacle 1080 to be vaporized. The vapor generated therefrom may then flow from the vaporizing chamber 1006, along the spiral flow path, to the suction opening 1009 for consumption by the user.

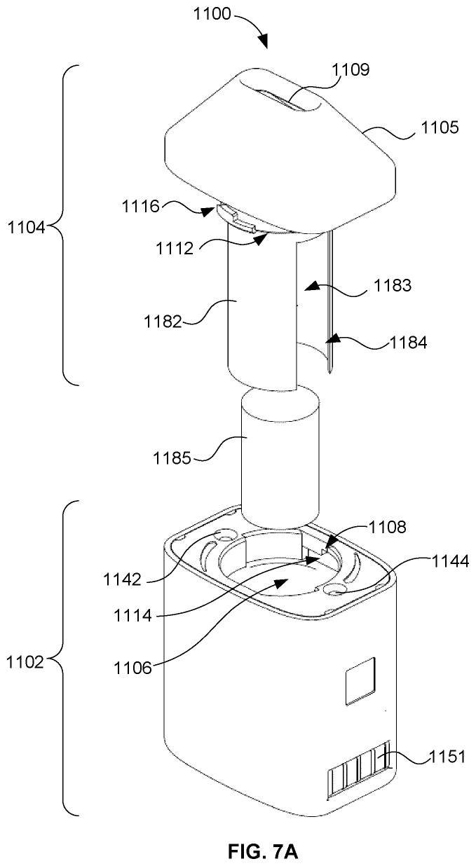

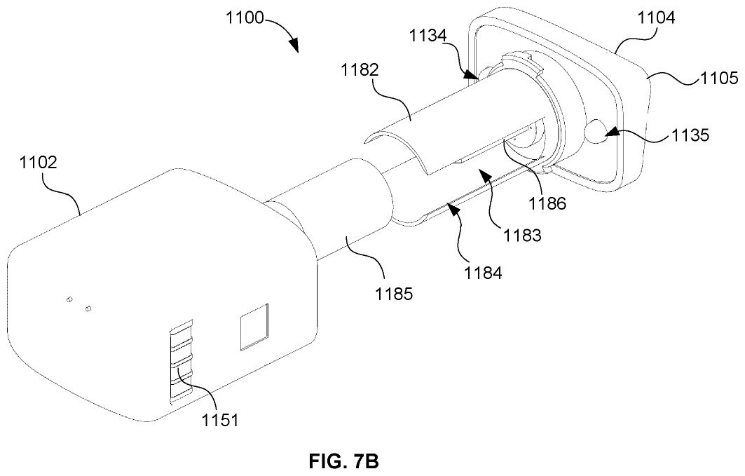

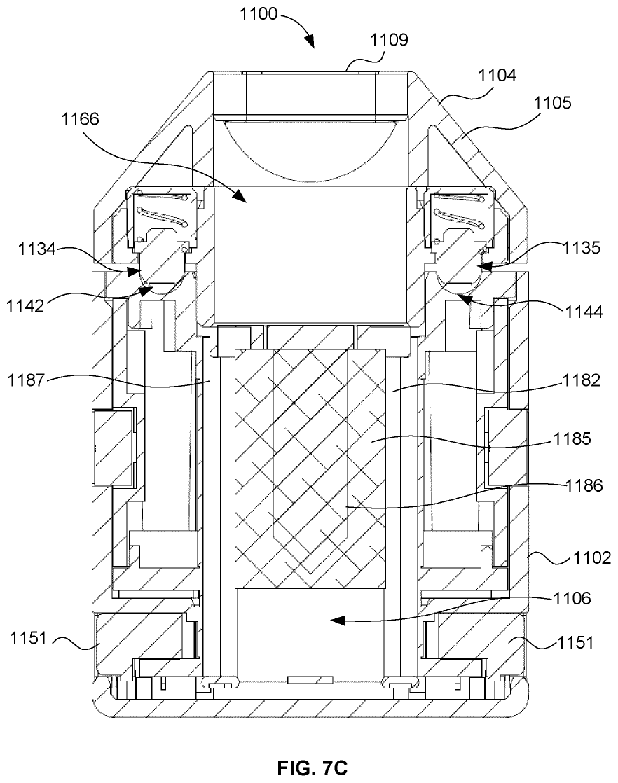

Another example vaporizer oven 1100 will be discussed with reference to FIGS. 7A to 7C. In this embodiment, the vaporizer oven 1100 may comprise a body 1102 and a closure member 1104.

The body 1102 may comprise a vaporizing chamber 1106 and an opening 1108 to the vaporizing chamber 1106. In some embodiments, the body 1102 may further comprise an insulating layer 1187 around the vaporizing chamber 1106 (shown in FIG. 7C). The insulating layer 1187 may comprise any suitable type of insulating material.

The closure member 1104 may removably engage the body to close the opening 1108. The closure member 1104 may comprise a mouthpiece portion 1105 and a suction opening 1109. The closure member 1104 may further comprise an internal conduit 1166 (shown in FIG. 7C) in fluid communication with the suction opening 1109 and the vaporizing chamber 1106 when the closure member 1104 is engaged with the body 1102.

The body 1102 may comprise a first locking component 1114. The closure member 1104 may comprise a second locking component 1116 configured to interlock with the first locking component 1114 when the closure member 1104 is engaged with the body 1102.

In some embodiments, the closure member 1104 may comprise a first biasing element 1134 and a second biasing element 1135 (shown in FIGS. 7B and 7C). The body 1102 may comprise a respective first recess 1142 and second recess 1144 (shown in FIGS. 7A and 7C) to at least partially receive the first and second biasing elements 1134 and 1135. The first and second biasing elements 1134 and 1135 may function in a similar manner to the first and second biasing elements 134 and 135 of the vaporizer oven 100.

The closure member 1104 may comprise a plug portion 1112 extending from the mouthpiece portion 1105. In this example, the closure member 1104 may also comprise a tubular extension 1182, extending from the plug portion 1112. The tubular extension 1182 may be configured to be received into the vaporizing chamber 1106 of the body 1102. In some embodiments, each of the tubular extension 1182 and the vaporizing chamber 1106 may be approximately cylindrical. In other embodiments, each of the tubular extension 1182 and the vaporizing chamber 1106 may be any other suitable complementary shape.

The tubular extension 1182 may comprise an internal chamber 1183 and a lateral opening 1184 to the internal chamber 1183. The internal chamber 1183 may be configured to receive a vaporing material therein. The vaporizing material may comprise dried plant material that has been formed into a plug, stick, or capsule, etc. An example plug 1185 is shown in FIGS. 7A to 7C.

In some embodiments, the closure member 1104 may further comprise a heating element 1186. In some embodiments, the heating element 1186 may extend from the mouthpiece portion 1105 into the internal chamber 1183 of the tubular extension 1182. In some embodiments, the heating element 1186 may be a knife-like heating element that pierces the plug 1185 when the plug 1185 is inserted into the internal chamber 1183.

In some embodiments, the heating element 1186 may be operatively connectable to a power source (not shown) in a vaporizer housing as described in more detail below. In some embodiments, the body 1102 may comprise one or more electrical connectors 1151 configured to engage a cooperating electrical connector(s) in the vaporizer housing to operably connect to the power source. In some embodiments, the first and second biasing elements 1134 and 1135 and the first and second recesses 1142 and 1144 may function as electrical contacts such that power may be transferred from the body 1102 to the heating element 1186 in the closure member 1104.

As shown in FIG. 7C, when the closure member 1104 is engaged with the body 1102, the tubular extension 1182 (having the plug 1185 therein) may be received into the vaporizing chamber 1106. The heating element 1186 may pierce the plug 1185. When the heating element 1186 is activated, the heating element 1186 may heat the plug 1185 to generate a vapor therefrom. The heating element 1186 may be configured to heat the plug 1185 without combusting the plant material therein such that the vaporizer oven 1100 may function in a similar manner to a “heat-not-burn” device.

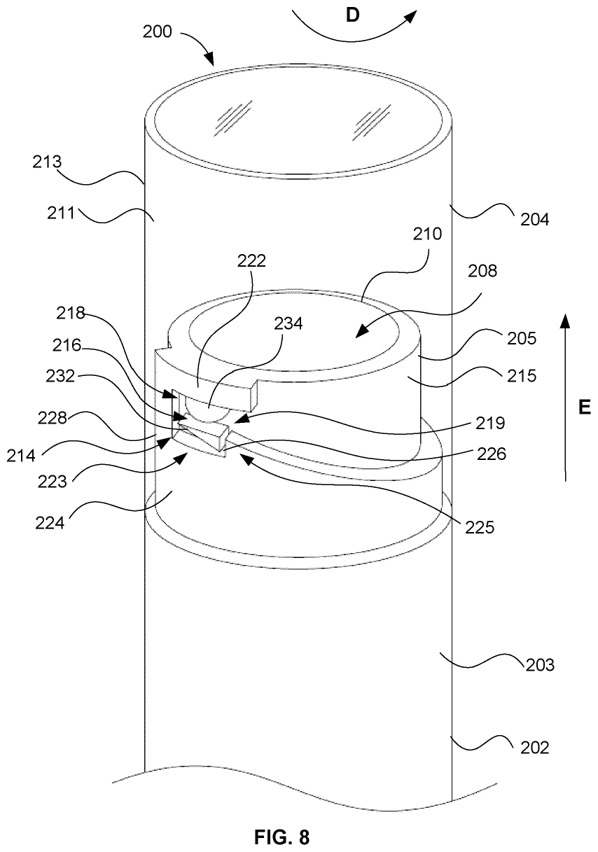

Another example vaporizer oven 200 will be discussed with reference to FIG. 8. In this embodiment, the vaporizer oven 200 may comprise a body 202 and a closure member 204. In FIG. 8, the closure member 204 is shown as transparent to better show the engagement of the closure member 204 and the body 202; however, the closure member 204 may be made of any suitable material and may be opaque.

The body 202 may comprise a main body portion 203 and an extended neck portion 205 extending therefrom. The main body portion 203 may define a vaporizing chamber therein (the vaporizing chamber is not visible in FIG. 8). The extended neck portion 205 may have an inner wall 210 and an outer wall 215, the inner wall 210 defining an opening 208 to the vaporizing chamber.

The closure member 204 may removably engage the body 202 to close the opening 208. The closure member 204 may comprise a suction opening (not shown) in fluid communication with the vaporizing chamber when the closure member 204 is engaged with the body 202. The closure member 204 may comprise an inner wall 211 and an outer wall 213.

The body 202 may comprise a first locking component 214 and the closure member 204 may comprise a second locking component 216 configured to interlock with the first locking component 214 when the closure member 204 is engaged with the body 202. The closure member 204 may therefore have a locked position and an unlocked position.

In this embodiment, the first locking component 214 may comprise a recess 218 defined by the outer wall 215 of the extended neck portion 205. The outer wall 215 may comprise a first, upper projection 222 proximal to the opening 208 and a second, lower projection 224 spaced apart from upper projection 222. The lower projection 224 may comprise a first section 223 and a second section 225, the second section 225 axially offset from the first section 223 and thereby forming a first rotational stop 226. A second rotational stop 228 may be disposed between upper and lower projections 222, 224. The outer wall 215 may thereby form an approximately C-shaped feature with the recess 218 being disposed between the first and second stops 226, 228 and an opening 219 provided to the recess 230.

The second locking component 216 may comprise a wedge-like projection 232 projecting from the inner wall 211 of the closure member 204. When the closure member 204 is engaged with the body 202, the wedge-like projection 232 may be received in the recess 218 between first and second stops 226, 228 to interlock the first and second locking components 214, 216. The upper and lower projections 222 and 224 of the first locking component 214 may function as upper and lower axial boundaries to limit the axial movement of the wedge-like projection 232 within the recess 230, thereby limiting the axial movement of the when the first and second locking components 214, 216 are interlocked.

In some embodiments, a biasing element 234 may be disposed in the body 202 to exert a biasing force to hold the wedge-like projection 232 of the closure member 204 in the recess 218 between the first and second stops 226, 228 when the closure member 204 is engaged with the body 202 in the locked position. In some embodiments, the biasing element 234 may comprise a spring. In other embodiments, the biasing element 234 may comprise any other suitable type of biasing element.

In some embodiments, the biasing element 234 may be disposed within the extended neck portion 205 of the body 202. In some embodiments, a medial space (not shown) may be defined between the outer wall 215 and the inner wall 210 of the extended neck portion 205 and the biasing element 234 may be disposed within the medial space. In some embodiments, an aperture (not shown) may extend from the medial space through the underside of the upper projection 222. At least a portion of the biasing element 234 may extend outward from the medial space through the aperture and project downward from the upper projection 222 towards the wedge-like projection 232. The biasing element 234 may exert a biasing force against the wedge-like projection 232 to hold the wedge-like projection 232 in the recess 230 between the first and second stops 226, 228. Therefore, when the closure member 204 is engaged with the body 202, the first stop 226 may prevent rotation of the closure member 204 with respect to the body 202 in the direction indicated by arrow D and the second stop 228 may prevent rotation in the opposite direction.

To disengage the closure member 204 from the body 202 in this embodiment, the closure member 204 may be moved axially away from the body 202 as indicated by arrow E to overcome the biasing force of the biasing element 234. Axial movement of the closure member 204 away from the body 202 may thereby compress the biasing element 234 and allow the wedge-like projection 232 to be displaced from first stop 226. The closure member 204 may then be rotated as indicated by arrow G to move the wedge-like projection 232 through the opening 219 of the recess 218, clearing the first stop 226 such that the closure member 204 is in the unlocked position. The closure member 204 may then be disengaged from the body 202 and removed.

Therefore, embodiments of the vaporizer ovens described herein may require two independent movements to disengage the closure member from the body, which could be difficult for a child or unauthorized user to perform. The vaporizer oven 100 may require a “push and twist” motion in which the closure member 104 is pushed towards the body 102 and then rotated with respect to the body 102 while continuing to push the closure member 104. The vaporizer ovens 1000 and 1100 may require a similar “push and twist” motion. The vaporizer oven 200 may require a “pull and twist” motion in which the closure member 104 is pulled away from the body 102 and then rotated with respect to the body 102 while continuing to pull the closure member 104. Embodiments of the vaporizer ovens described herein thus can decrease the possibility of a child or an unauthorized user opening the vaporizer oven and accessing the vaporization materials contained therein. Embodiments of the vaporizer ovens may also prevent inadvertent opening of vaporizer chamber by the user during or between “vape” sessions.

Other variations are also possible. In another alternative embodiment, a vaporizer oven may comprise a closure member having a first locking component, a body having a second locking component, and a depressible member such as a push button received in an aperture extending through the closure member or the body. The depressible member may be positioned adjacent to a biasing element such that depression of the depressible member through the aperture compresses the biasing element to allow the first and second locking components to be disengaged.

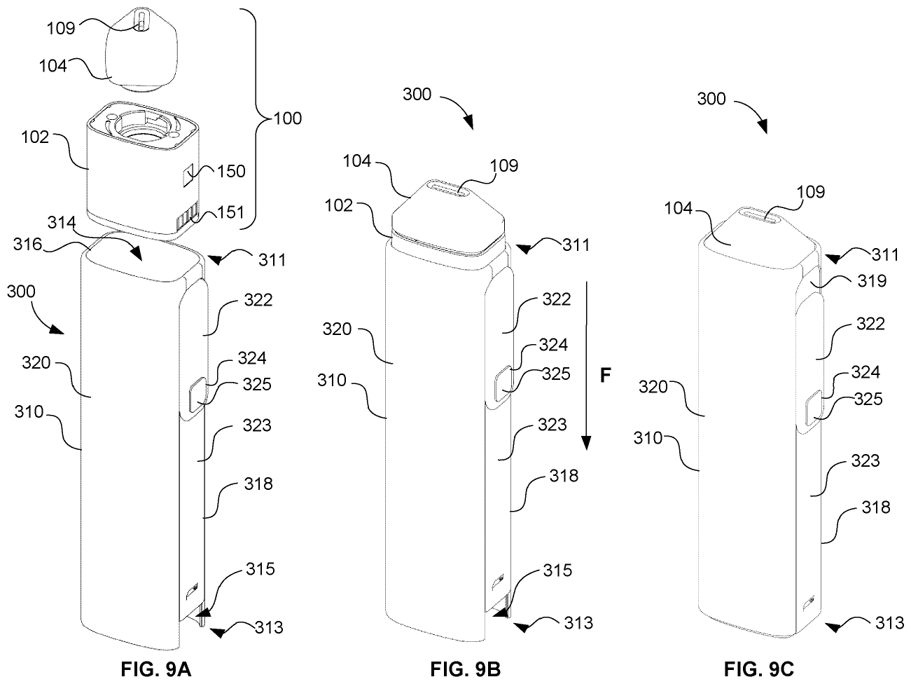

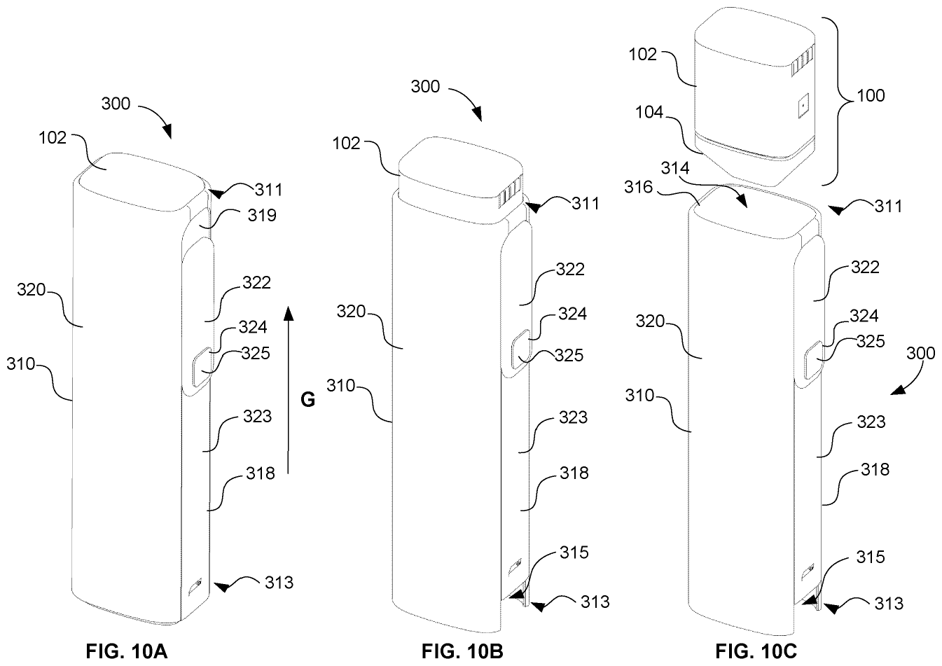

An example vaporizer 300, including the vaporizer oven 100, will now be discussed with reference to FIGS. 9A to 9C and FIGS. 10A to 10C. The vaporizer 300 in this embodiment may comprise the vaporizer oven 100 and a vaporizer housing 310 configured to engage the vaporizer oven 100. Although this example shows vaporizer housing 310 configured to engage the vaporizer oven 100, in other embodiments, vaporizer housing 310 may be configured to engage the vaporizer oven 1000, 1100, or 200, or any other embodiment of a vaporizer oven.

The housing 310 may have a first end 311 and a second end 313. As shown in FIGS. 9A and 10C, the housing 310 may comprise a cavity 314 at the first end 311 that at least partially receives the vaporizer oven 100. The cavity 314 may be any suitable shape and size to receive the vaporizer oven 100. In some embodiments, both the vaporizer oven 100 and the cavity 314 of the housing 310 may have non-circular cross-sections in the width-wise direction such that the vaporizer oven 100 cannot be rotated when received into the cavity 314. In other embodiments, the vaporizer oven 100 and the housing 310 may have approximately circular cross-sections such that the vaporizer oven 100 is rotatable within the housing 310.

As shown in FIG. 9B to 9C, the vaporizer oven 100 may be received into the cavity 314 of the housing 310 with the closure member 104 directed outward, away from the cavity 314. The closure member 104 may therefore form a terminal end of vaporizer 300. FIG. 9B shows the vaporizer oven 100 partially received into cavity 314 and FIG. 9C shows the vaporizer oven 100 fully received into cavity 314. When the vaporizer oven 100 is fully received into cavity 314 with the closure member 104 directed outward, the vaporizer oven 100 may be in an operational position. As used herein, the term “operational position” may refer to the position of the vaporizer oven 100 in which the vaporizer 300 is ready, or in condition, to be used for “vaping” by a user.

In some embodiments, the vaporizer 300 may further comprise a power source, such as a battery (not shown), operable to connect to the heating means of the vaporizer oven 100 when the vaporizer oven is in the operational position. In some embodiments, the vaporizer 300 may also comprise a control unit (not shown). The control unit may comprise a processor operatively connected to the power source and to the heating means of the vaporizer oven 100 when the vaporizer oven 100 is in the operational position. The power source and the control unit may be disposed within the housing 310, below the cavity 314.

In some embodiments, the housing 310 may comprise one or more operative connection elements (e.g. electrical contacts, mechanical connectors, etc.) within the cavity 314 that engage corresponding operative connection elements in the vaporizer oven 100 when the vaporizer oven 100 is in the operational position. Electrical contacts may be in the form of conductive strips or pads, male or female plug elements, or any other conductive contact point suitable for engaging another contact and forming an electrical connection. For example, the vaporizer oven 100 may comprise the electrical connectors 151 as shown in FIG. 5. Thus, electrical power and/or control signals may be transferred from the housing 310 to the vaporizer oven 100 or vice versa when the vaporizer oven 100 is in the operational position. In some embodiments, where the vaporizer oven 100 has an approximately circular cross-section, the operative connection elements may be configured such that the vaporizer oven 100 can be electrically connected to the housing 310 in more than one rotational position.

Referring to FIGS. 10A to 10C, the vaporizer oven 100 may also be received into the cavity 314 of the housing 310 with the closure member 104 directed inward towards the interior of the cavity 314. The body 102 may therefore comprise a terminal end of vaporizer 300. FIG. 10A shows the vaporizer oven 100 partially received into cavity 314 and FIG. 10B shows the vaporizer oven 100 fully received into the cavity 314. When the vaporizer oven 100 is fully received into the cavity 314, with the closure member 104 directed inward, the vaporizer oven 100 may be in a non-operational position. As used herein, the term “non-operational position” may refer to the position of the vaporizer oven 100 in which the vaporizer 300 is not functional or ready for use. In some embodiments, the non-operational position may be axially reversed relative to the operational position. In the non-operational position, the vaporizer 300 may be relatively convenient to store or transport between vaping sessions. As shown in FIG. 10C, the vaporizer oven 100 may then be removed from the cavity 314 and re-inserted into the operational position as discussed above (and vice versa).

As shown in FIG. 9A and FIG. 10C, cavity 314 in this embodiment may be at least partially defined by an inner wall 316 of the housing 310. As shown in FIGS. 9B to 9C and 10A to 10B, the inner wall 316 may cover at least a portion of the vaporizer oven 100 when the vaporizer oven 100 is received into the cavity 314.

As shown in FIG. 9C, when the vaporizer oven 100 is in an operational position, the inner wall 316 may at least partially cover the closure member 104. In this example, the inner wall 316 may at least partially cover the base portion 105 of the closure member 104. When at least a portion of the closure member 104 is covered by the inner wall 316, the closure member 104 cannot be rotated with respect to the body 102 (as indicated by arrow B in FIG. 1C) and thus the closure member 104 cannot be disengaged from the body 102. Thus, the opening 108 to the vaporizing chamber 106 may not be accessible when the vaporizer oven 100 is in the operational position.

As shown in FIG. 10A, when the vaporizer oven 100 is in a non-operational position, the closure member 104 may be fully covered by the inner wall 316 and the body 102 may also be partially covered by the inner wall 316. Thus, the opening 108 to the vaporizing chamber 106 may also not be accessible when the vaporizer oven 100 is in a non-operational position.

Therefore, in some embodiments, accessing the vaporizing chamber 106 of the vaporizer oven 100 and any vaporizing material contained therein, may require removing the vaporizer oven 100 from the housing 310 and then disengaging the closure member 104 from the body 102 as discussed above. This combination of actions may thus make it much more difficult for a child or an unauthorized user to open the vaporizer oven 100 to access the vaporizing material therein or for the user to inadvertently open the vaporizer oven 100 during or between “vape” sessions.

In some embodiments, the housing 310 may further comprise a slidable sidewall 318, slidably engaged with adjacent sidewalls 320, to facilitate removal and re-insertion of the vaporizer oven 100. In some embodiments, the slidable sidewall 318 is a metal slider. In some embodiments, the slidable sidewall 318 may include a raised portion 322 and a lower portion 323. The user may place their thumb or finger on the lower portion 323, abutting the raised portion 322, to move the slidable sidewall 318 longitudinally with respect to the other sidewalls.

In some embodiments, the slidable sidewall 318 may comprise an internal projection (not shown) projecting into the cavity 314. The vaporizer oven 100 in these embodiments abuts the internal projection when the vaporizer oven 100 is fully received into the cavity 314. Therefore, in some embodiments, sliding the slidable sidewall 318 with respect to adjacent sidewalls 320 can also slide the vaporizer oven 100 longitudinally with respect to the housing 310.

In other embodiments, the slidable sidewall 318 may be coupled to at least one of the power source and the control unit housed within the housing 310. When the vaporizer oven 100 is fully received into the cavity 314 and operatively connected to the power source and the control unit, the vaporizer oven 100 can abut at least one of the power source and the control unit. In this embodiment, sliding the slidable sidewall 318 with respect to adjacent sidewalls 320 may also slide at least one of the power source and control unit and thus can also slide the vaporizer oven 100 longitudinally with respect to the housing 310.

As shown in FIG. 9A, the slidable sidewall 318 may have a first position in which the slidable sidewall 318 is longitudinally shifted towards the first end 311 of the housing 310 such that a gap 315 is present at the second end 313 of the housing 310. The slidable sidewall 318 may be in the first position prior to insertion of the vaporizer oven 100 into the cavity 314 of the housing 310. As shown in FIG. 9B, once the vaporizer oven 100 is partially inserted into the cavity 314, the slidable sidewall 318 may be slid longitudinally downward as indicated by arrow F until the vaporizer oven 100 is fully inserted into the cavity 314. As shown in FIG. 9C, the slidable sidewall 318 may then be in a second position in which the slidable sidewall 318 can be shifted longitudinally towards the second end 313 of the housing 310 such that the gap 315 is no longer present and a recess 319 is exposed proximate the first end 311 of the housing.

As shown in FIGS. 10A-10C, the slidable sidewall 318 may also be slid longitudinally upward (as indicated by arrow G) from the second position to the first position such that the slidable sidewall 318 is received into the recess 319. As the slidable sidewall 318 is slid upward, the vaporizer oven 100 is slid at least partially out of the cavity 314 to allow the user to readily grip the vaporizer oven 100 and remove it from the housing 310.

In some embodiments, the vaporizer oven 100 may further comprise at least one magnet 150 and the inner wall 316 of the housing 310 may further comprise at least one magnet of opposite polarity (not shown) such that a magnetic force is generated between the magnets when the vaporizer oven 100 is received into the cavity 314 of the housing 310 in the operational position. The magnetic force may help secure the vaporizer oven 100 in the housing 310 and may prevent the vaporizer oven 100 from inadvertently sliding out of cavity 314 while in use.

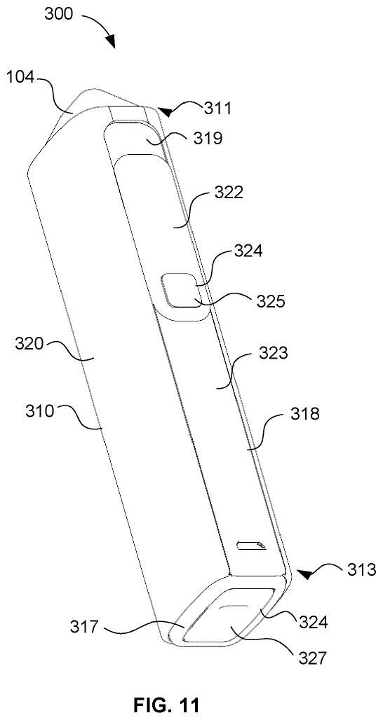

As shown in FIG. 11, in some embodiments, the housing 310 may further comprise one or more user interface elements 324 operatively connected to the processor of the control unit. The processor may thereby receive input from the user interface elements 324 to activate the heating means by supplying power thereto. In this example, the user interface elements 324 may comprise a first button 325 on the side of the housing 310 (more particularly on the slidable sidewall 318 as discussed below) and a second button on a bottom surface 317 at the second end 311 of the housing 310. In some embodiments, the first button 325 may be used to control activation of the heating means of the vaporizer oven 100. The second button 327 may be used alone or in combination with the first button 325 to program and/or change settings, etc. of the heating means. In some embodiments, activation of the heating means of the vaporizer oven 100 may require the user to press both buttons 325, 327 simultaneously or in a particular sequence, thereby providing an additional safety feature to prevent use by a child or unauthorized user.

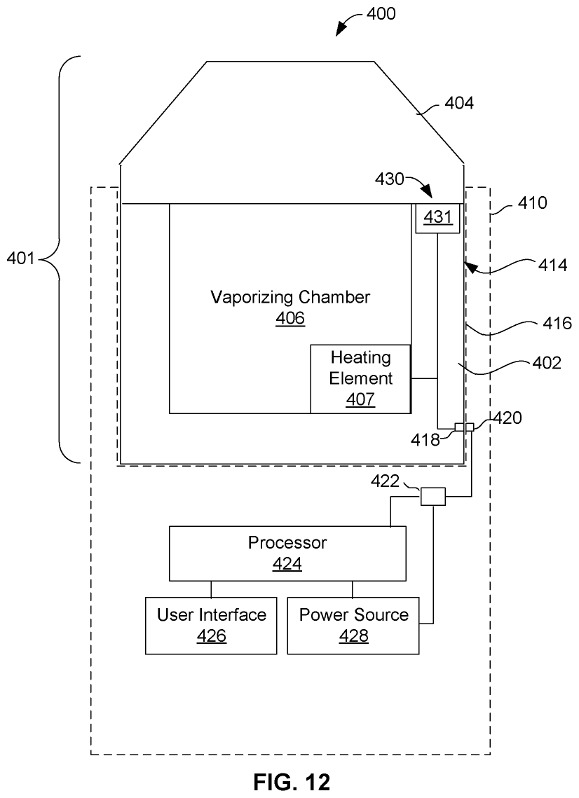

FIG. 12 is a functional block diagram of another example vaporizer 400 according to some embodiments. The vaporizer 400 may comprise a vaporizer oven 401 and a housing 410. The vaporizer oven 401 may comprise a body 402 and a closure member 404 that engages the body 402. In some embodiments, the body 402 and the closure member 404 may each comprise respective locking components (not shown) as described above. Although, in this embodiment, the vaporizer oven 401 in FIG. 12 is similar to the vaporizer oven 100 of FIGS. 1A-5, in other embodiments, the vaporizer oven 401 may be similar to the vaporizer oven 1000, 1100, or 200, or any other embodiment of the vaporizer ovens disclosed herein.

The housing 410 may comprise a cavity 414 defined by an inner wall 416. The vaporizer oven 401 may be at least partially received into the cavity 414. Note that the housing 410 is shown in dashed lines for illustrative purposes only to distinguish the housing 410 from the vaporizer oven 401. In some embodiments, the housing 410 may receive the vaporizer oven 401 in either an operational position (as shown in FIG. 12) or a non-operational position.

The body 402 of the vaporizer oven 401 may comprise a vaporizing chamber 406 and a heating element 407 operatively connected to the vaporizing chamber 406 and operable to heat the vaporizing material therein. In other embodiments, the closure member 404 may comprise the heating element 407 such as, for example, in a similar manner to the vaporizer oven 1100 as described above. In some embodiments, the heating element 407 may be positioned at least partially within the vaporizing chamber 406. The heating element 407 may be, for example, a coiled filament or any other suitable element for generating heat to vaporize the material within the chamber 406.

The housing 410 may comprise a power source 428. In some embodiments, the power source 428 comprises a battery. In other embodiments, the power source 428 may comprise any other suitable power source. The power source 428 may be operatively connected to the heating element 407. The housing 410 may further comprise a processor 424 and at least one user interface element 426. The one or more user interface elements 426 may comprise, for example, a control such as a button. The power source 428 and user interface elements 426 may be operatively connected to the processor 424.

The processor may process input from the user interface elements 426 and activate the heating element 407 by providing power from the power source 428 to the heating element 407 accordingly. In some embodiments, a switch 422 may be provided in the housing 410. In other embodiments, the switch 422 may be provided in the vaporizer oven 401. The switch 422 may be activated by the processor 424 to provide power to the heating element 407.

The vaporizer oven 401 may comprise a first operative connection element 418 (such as an electrical connector). The housing 410 may comprise a second operative connection element 420 (such as an electrical connector). The first and second operative connection elements may engage each other as shown in FIG. 12 when the vaporizer oven 401 is in the operational position. The first and second operative connection elements 418 and 420 may provide an electrical connection between the vaporizer oven 401 and the housing 410 to allow power to be transferred to the heating element 407 from the power source 428. Removing the vaporizer oven 401 from the operational position may disengage the operative connection elements 418 and 420 such that the heating element 407 is disabled. Although only one set of operative connection elements 418, 420 is shown in this embodiment, it will be understood that any suitable number of operative connection elements may be provided.

In some embodiments, the vaporizer oven 400 may further comprise at least one activation control mechanism 430 that disables activation of the heating element 407 when the closure member 404 is disengaged from the body 402 of the vaporizer oven 401. In this example, the activation control mechanism 430 is shown disposed in the body 402; however, the activation control mechanism 430 may alternatively be disposed in the closure member 404. In some embodiments, the activation control mechanism 430 may comprise a sensor 431 that senses when the closure member 404 is disengaged from the body 402 or rotated away from the locked position. The processor 424 may be operatively connected to the sensor 431 to receive output therefrom and disable or enable activation of the heating element 407 responsive to the output from the sensor 431.

Therefore, in some embodiments, the activation control mechanism 430 may prevent activation of the vaporizer oven 401 by a child or other unauthorized user or inadvertent activation by the user in between vaping sessions when the closure member 404 is disengaged from the body 402.

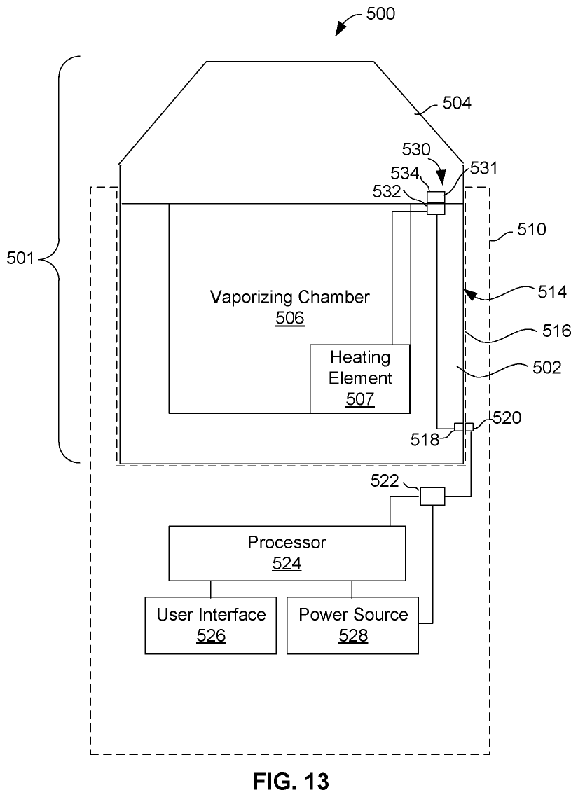

FIG. 13 is a functional block diagram of another example vaporizer 500 according to some embodiments.

The vaporizer 500 may comprise a vaporizer oven 501 and a housing 510. The vaporizer oven 501 may comprise a body 502 and a closure member 504. The body 502 may comprise a vaporizing chamber 506 and a heating element 507. In some embodiments, the body 502 and the closure member 504 may each comprise respective locking components (not shown) as described above.

The housing 510 may comprise a cavity 514 defined by an inner wall 516 that at least partially receives the vaporizer oven 501. The housing 510 may receive the vaporizer oven 501 in either an operational position (as shown in FIG. 13) or a non-operational position.

The housing 510 may comprise a processor 524, at least one user interface element 526, and a power source 528, similar to the processor 424, user interface elements 426, and power source 428 of the vaporizer 400 of FIG. 12 as discussed above. The processor may process input from the user interface elements 526 and activate a switch 522 to provide power to the heating element 507.

The vaporizer oven 501 may comprise a first operative connection element 518 and the housing 510 may comprise a second operative connection element 520 that may engage one another when the vaporizer oven 501 is in the operational position.

The vaporizer oven 501 may further comprise at least one activation control mechanism 530. In this example, the activation control mechanism 530 may comprise an electrical switch 531 that is open when the closure member 504 is disengaged from the body 502 and closed when the closure member 504 engages the body 502. In some embodiments, the switch 531 may comprise a biasing element 534 in one of the body 502 and the closure member 504 and an electrical contact 532 in the other of the body 502 and the closure member 504. The biasing element 534 may be similar to the biasing elements 134/135 or 234 of the vaporizer ovens 100 and 200 as described above. The biasing element 534 may contact the electrical contact 532, closing the switch, when the closure member 504 engages the body 502. The switch 531 may be open when the closure member 504 is removed or rotated away from the locked position and the biasing element 534 is no longer in contact with the electrical contact 532.

In other embodiments, the switch 531 may comprise any suitable combination of electrical contacts in the body 502 and the closure member 504.

Other variations are possible. In some embodiments, the activation control mechanism may not be dependent on the presence of locking components and/or a biasing element. Thus, embodiments of vaporizer ovens may be provided comprising at least one activation control mechanism without first and second locking components and/or a biasing element.

A method for making a vaporizer oven is also provided. The method may be used to make any embodiment of vaporizer ovens, such as vaporizer ovens 100, 1000, 1100, 200, 401, and 501 as described herein.

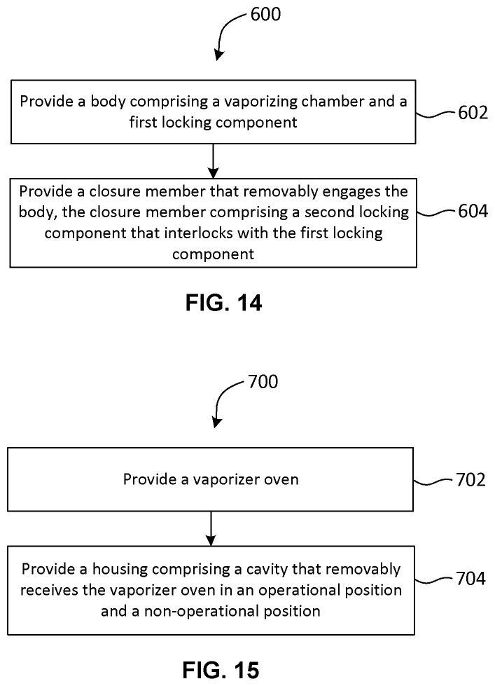

FIG. 14 is a flowchart of an example method 600 for making a vaporizer oven according to some embodiments.

At block 602, a body may be provided. The body may comprise a vaporizing chamber and a first locking component. At block 604, a closure member may be provided that removably engages the body. The closure member may comprise a second locking component that interlocks with the first locking component. The closure member may be removable from the body and movable between a locked position and an unlocked position as described above. The term “providing” in this context may refer to making, manufacturing, buying, acquiring, or otherwise obtaining the body and the closure member.

The method 600 may further comprise engaging the closure member with the body in either a locked position or unlocked position. The method 600 may further comprise moving the closure member from a locked position to unlocked position (or vice versa). In some embodiments, moving the closure member from a locked position to unlocked position (or vice versa) may comprise rotating the closure member with respect to the body. Alternatively, the body could be rotated with respect to the closure member. In some embodiments, moving the closure member from a locked position to unlocked position (or vice versa) may comprise first axially moving the closure member towards or away from the body, followed by rotating the closure member with respect to the body. Alternatively, the body could be moved axially away from or towards the closure member and then rotated with respect to the closure member.

In some embodiments, providing the body may further comprise integrally forming the first locking component. In some embodiments, providing the closure member may further comprise integrally forming the second locking component. In some embodiments, providing the body may further comprise placing a heating means in the body (e.g. in the vaporizing chamber). In some embodiments, providing the closure member further comprises forming a mouthpiece portion of the closure member and forming a suction opening therethrough.

In some embodiments, providing the body and the closure member may further comprise providing at least one biasing element disposed in the body and/or the closure member. In some embodiments, providing the body and the closure member may further comprise providing at least one activation control mechanism in the body and/or closure member.

A method for making a vaporizer is also provided herein. The method may be used to make any embodiment of the vaporizers 300, 400, and 500 as described herein.

FIG. 15 is a flowchart of an example method 700 for making a vaporizer according to some embodiment.

At block 702, a vaporizer oven may be provided. The vaporizer oven may comprise a body and a closure member that engages the body. The vaporizer oven may comprise any of the features of the example vaporizer ovens described above. At block 704, a housing may be provided. The housing may comprise a cavity that removably receives the vaporizer oven in an operational position and a non-operational position. The term “providing” in this context may refer to making, manufacturing, buying, acquiring, or otherwise obtaining the vaporizer oven and the housing.

The method 700 may further comprise inserting the vaporizer oven into the cavity in either an operational or non-operation position. The method 700 may further comprise moving the vaporizer oven from the operation position to the non-operational position (or vice versa). In some embodiments, moving the vaporizer oven from the operation position to the non-operational position (or vice versa) can comprise removing the vaporizer oven from the cavity of the housing, axially reversing the vaporizer oven, and re-inserting it into the cavity in the other position.

In some embodiments, the housing may comprise a slidable sidewall and removing the vaporizer oven from the housing may comprise longitudinally sliding the vaporizer oven out of the cavity using the slidable sidewall. The vaporizer oven may be reinserted into the cavity by engaging the vaporizer oven with the slidable sidewall and longitudinally sliding the vaporizer oven into the cavity.

In some embodiments, providing the housing may further comprise providing a power source and a control unit (e.g. a processor) within the housing. In some embodiments, the method 700 may further comprise powering the vaporizer oven when the vaporizer oven is received into the cavity in an operational position. In some embodiments, the method 700 may further comprise controlling the power to the vaporizer oven based on input from a user and/or based on the output of at least one activation control mechanism in the vaporizer oven.

It should be apparent to those skilled in the art that more modifications besides those already described are possible without departing from the inventive concepts herein. The inventive subject matter, therefore, is not to be restricted except in the scope of the disclosure. Moreover, in interpreting the disclosure, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced.

Although particular embodiments have been shown and described, it will be appreciated by those skilled in the art that various changes and modifications might be made without departing from the scope of the invention. The terms and expressions used in the preceding specification have been used herein as terms of description and not of limitation, and there is no intention in the use of such terms and expressions of excluding equivalents of the features shown and described or portions thereof, it being recognized that the invention is defined and limited only by the claims that follow.

It is to be understood that a combination of more than one of the approaches described above may be implemented. Embodiments are not limited to any particular one or more of the approaches, methods or apparatuses disclosed herein.

One skilled in the art will appreciate that variations, alterations of the embodiments described herein may be made in various implementations without departing from the scope of the claims.

1. A vaporizer oven comprising:
a body comprising a vaporizing chamber, an opening to the vaporizing chamber, and a first locking component; and
a removable closure member that removably engages the body to close the opening, the closure member comprising a second locking component that interlocks with the first locking component when the closure member engages the body;
wherein the closure member is movable when engaged to the body between a locked position in which the first and second locking components are interlocked and the closure member is secured to the body, and an unlocked position in which the first and second locking components are disengaged and the closure member is removable from the body.

2. The vaporizer oven of claim 1, wherein one of the first and second locking components comprises a projection and the other of the first and second locking components comprises a recess, the projection being received into the recess to interlock the first and second locking components when the closure member engages the body in the locked position.

3. The vaporizer oven of claim 2, wherein the closure member rotatably engages the body and wherein the closure member is rotatable between a first rotational position in which the projection is received within the recess and the first and second locking components are interlocked, and a second rotational position in which the projection is displaced from the recess and the first and second locking components are disengaged.

4. The vaporizer oven of claim 3, wherein the recess comprises at least one axial boundary that limits axial movement of the projection when the first and second locking components are interlocked, thereby limiting axial movement of the closure member away from the body when the closure member is in the first rotational position.

5. The vaporizer oven of claim 4, further comprising at least one biasing element disposed in one of the body and the closure member, the at least one biasing element exerting a biasing force to maintain the closure member in the first rotational position.

6. The vaporizer oven of claim 5, wherein at least a portion of the recess is disposed between a first rotational stop and a second rotational stop; and wherein the biasing force of the biasing element holds the projection within the recess between the first and second rotational stops to maintain the closure member in the first rotational position.

7. (canceled)

8. (canceled)

9. The vaporizer oven of claim 1, wherein the first locking component is integral to the body and the second locking component is integral to the closure member.

10. The vaporizer oven of claim 1, wherein the closure member comprises a mouthpiece portion having a suction opening, the suction opening in fluid communication with the vaporizing chamber when the closure member is engaged with the body.

11. The vaporizer oven of claim 10, wherein the closure member further comprises an inner sleeve defining an internal conduit therethrough, the internal conduit in fluid communication with the suction opening and the vaporizing chamber when the closure member is engaged with the body.

12. The vaporizer oven of claim 11, wherein the closure member further comprises a helical insert received within the internal conduit, the helical insert defining approximately spiral airflow path between the vaporizing chamber and the suction opening.

13. The vaporizer oven of claim 11, wherein the inner sleeve is colored to indicate a characteristic of a vaporized material to be vaporized in the vaporizing chamber.

14. The vaporizer oven of claim 1, further comprising a heating means operatively connected to the vaporizing chamber, the heating means activatable to heat the vaporizing chamber.

15. The vaporizer oven of claim 14, further comprising an activation control mechanism, operatively connected to the heating means, wherein the activation control mechanism disables activation of the heating means when the closure member is disengaged from the body.

16. (canceled)

17. (canceled)

18. A vaporizer comprising:
the vaporizer oven of claim 1; and
a housing that engages the vaporizer oven, the housing having a cavity that at least partially receives the vaporizer oven.

19. The vaporizer of claim 18, wherein the cavity is defined by an inner wall that covers at least a portion of the vaporizer oven when the vaporizer oven is at least partially received into the cavity.

20. The vaporizer of claim 19, wherein the inner wall of the cavity covers at least a portion of the closure member of the vaporizer oven to prevent movement of the closure member between the locked and unlocked positions.

21. The vaporizer of claim 18, wherein the housing further comprises a slidable sidewall that engages the vaporizer oven to longitudinally slide the vaporizer oven into and out of the cavity.

22. The vaporizer of claim 18, wherein the housing is configured to receive the vaporizer oven into the cavity with the vaporizer oven in an operational position in which the closure member is directed outward from the cavity, and receive the vaporizer oven into the cavity with the vaporizer oven in a non-operational position in which the closure member is directed inward into the cavity.

23. (canceled)

24. (canceled)

25. A method for making a vaporizer, comprising:
providing a vaporizer oven comprising a body having a vaporizer chamber and a closure member that engages the body; and
providing a housing comprising a cavity that removably receives the vaporizer oven in an operational position and a non-operational position.

26. The method of claim 25, further comprising inserting the vaporizer oven into the cavity in one of the operation position and the non-operational position, wherein inserting the vaporizer oven comprises sliding the vaporizer oven longitudinally with respect to the housing.
